(19) United States
(12) Patent Application Publication
(10) Pub. No.: US 2006/0111730 A1
(43) Pub. Date: May 25, 2006

(54) Deformity reduction instrument and method

(75) Inventor: James Scott Hay, Parkland, FL (US)

Correspondence Address: John Christopher;Christopher & Weisberg, P.A., Suite 2040, 200 East Las Olas Boulevard, Fort Lauderdale, FL, 33301, US

(73) Assignee: Medical Innovators, Inc.

(21) Appl. No.: 11/294,886

(22) Filed: Nov. 21, 2005

Related U.S. Application Data
(60) Provisional application No. 60/630,467, filed on Nov. 23, 2004.

(51) Int. Cl. A61F 2/36

(52) U.S. Cl. 606/104

(57) ABSTRACT

The present invention provides a medical device for reducing a rod towards a bone fastener, the medical device having a shaft defining a proximal end and a distal end, where the shaft further defines a first threaded portion. A gripper assembly may be coupled to the distal end of the shaft, where the gripper assembly is engageable with the bone fastener. The medical device also includes a slide element slideably disposed on the shaft and coupled to the gripper assembly, with the slide element defining a second threaded portion. A pusher element is slideably coupled to shaft, whereby the pusher element defines an aperture engageable with the rod. A shaft grip can be rotatably engaged with the first threaded portion of the shaft, and the shaft grip can abut at least a portion of the pusher element. The medical device can also include a jaw nut coupled to the shaft, with the jaw nut rotatably engaging the second threaded portion of the slide element, as well as a lever assembly pivotably coupled to the shaft, the lever assembly defining a rod channel engageable with the rod, and further including a latch mechanism for releasably securing the lever assembly to the shaft.

### CROSS-REFERENCE TO RELATED APPLICATION

This application is related to and claims priority to U.S. Provisional Patent Application Ser. No. 60/630,467, filed Nov. 23, 2004, entitled DEFORMITY REDUCTION INSTRUMENT AND METHOD, the entirety of which is incorporated herein by reference.

### STATEMENT REGARDING FEDERALLY SPONSORED RESEARCH OR DEVELOPMENT

n/a

### FIELD OF THE INVENTION

The present invention relates generally to surgical instrumentation for moving one part of a surgical implant into adjacent position or contact with another surgical implant, and more particularly, to an instrument for securing a spinal rod to a bone fixation element.

### BACKGROUND OF THE INVENTION

There are many types of spinal column disorders, including scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease or trauma, such as ruptured or slipped discs, degenerative disc disease, fractured vertebra, and the like. Patients that suffer from such conditions usually experience extreme and debilitating pain, as well as diminished nerve function.

Surgical techniques commonly referred to as spinal fixation use surgical implants and/or mechanical immobilization to fuse two or more vertebral bodies of the spinal column. Spinal fixation may also be used to alter the alignment of adjacent vertebral bodies relative to one another so as to change the overall alignment of the spinal column. Such techniques have been used effectively to treat the above-described conditions and, in many cases, to relieve pain.

One spinal fixation technique involves immobilizing the spine using orthopedic stabilizing rods, commonly referred to as spine rods, which are positioned generally parallel to the spine. Exposing the spine posteriorly and fastening bone screws to the pedicles of vertebral bodies may accomplish this. The pedicle screws are generally placed two per vertebra and serve as anchor points for the spine rods. Implants adapted for receiving a spine rod therethrough can be used to join the spine rods to the pedicle screws. In one example, a setscrew or fastener then fastens the spine rod into a seat or receptacle of the implants.

Enormous force is often required to join the implanted spinal screws or hooks (implants) to the rod. To do this, a surgeon requires the ability to move and control both the rod and the spinal implant simultaneously, each in an independent multiplanar fashion until the rod receptacle of the implant captures the rod. The rod and the implant can be pulled to approximation (commonly referred to as “reducing” the rod or rod reduction) independently in 3 dimensions until their orientation in the X, Y and Z planes match for adjoining the rod to the implant via the receptacle. However, current devices work in only a single plane, which requires significantly more rod contouring in an effort to match two of the three planes prior to attempting reduction of the rod to the spinal implant or reduction of the spinal implant to join the rod.

### SUMMARY OF THE INVENTION

The present invention advantageously provides instrumentation for rod reduction during orthopedic surgery that is efficient, convenient to use and provide for simultaneous manipulation of a rod and a spinal implant. In this regard, the apparatus has a shaft assembly and a lever assembly. The shaft assembly can include a shaft defining a proximal end, a distal end, a top portion, and a bottom portion, with the shaft further defining a first threaded portion. A gripper assembly may be coupled to the distal end of the shaft, with the gripper assembly being engageable with the bone fastener. The medical device may further provide a slide element slideably disposed on the top portion of the shaft and coupled to the gripper assembly, with the slide element defining a second threaded portion. A pusher element may be slideably coupled to the bottom portion of the shaft, whereby the pusher element defines an aperture engageable with the rod. Further, a shaft grip can be provided that is rotatably engaged with the first threaded portion of the shaft such that the shaft grip abuts at least a portion of the pusher element. In addition, a jaw nut can be coupled to the shaft such that jaw nut is rotatably engaged with the second threaded portion of the slide element.

The present invention may further include a lever assembly pivotably coupled to the shaft, with the lever assembly further being slideably positionable on the shaft. The lever assembly can include a lever element having proximal and distal ends and a lever grip coupled to the proximal end of the lever. The lever element can further include two lever arms, each having a distal portion where each lever includes a rod channel configured to receive a rod therein, and may further include a latch mechanism for releasably securing the lever assembly to the shaft.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein like designations refer to like elements, and wherein:

FIG. 1 is a perspective view of an embodiment of a medical device in accordance with the present invention;

FIG. 2 is a top view of an embodiment of the medical device of FIG. 1;

FIG. 3 is a side view of an embodiment of the medical device of FIG. 1;

FIG. 4 is an assembly view of the medical device of FIG. 1;

FIG. 5 is a perspective view of an embodiment of a shaft assembly of a medical device in accordance with the present invention; and

FIG. 6 is an illustration of an embodiment of a medical device in accordance with the present invention.

### DETAILED DESCRIPTION OF THE INVENTION

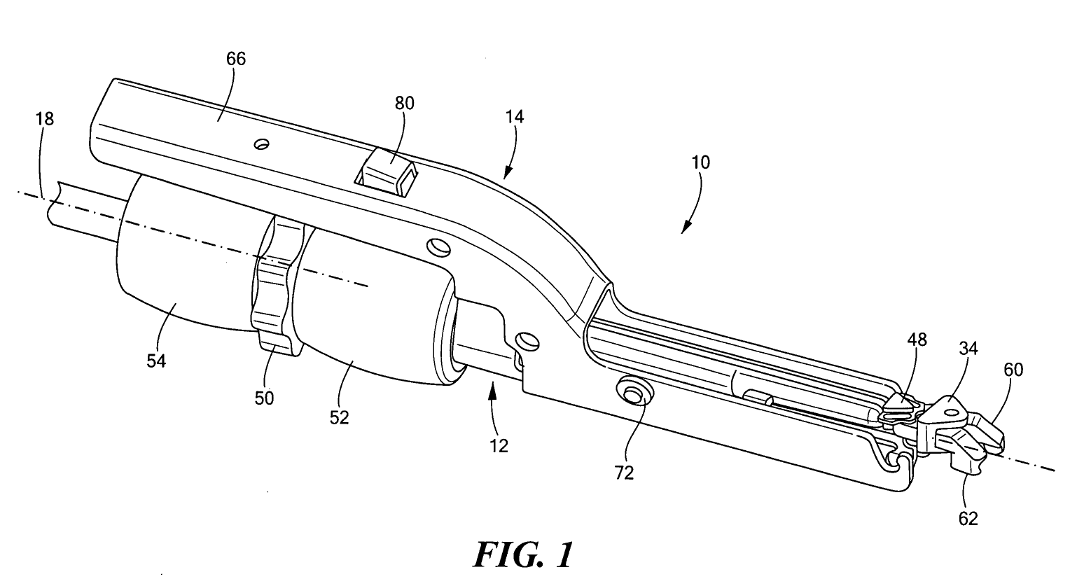

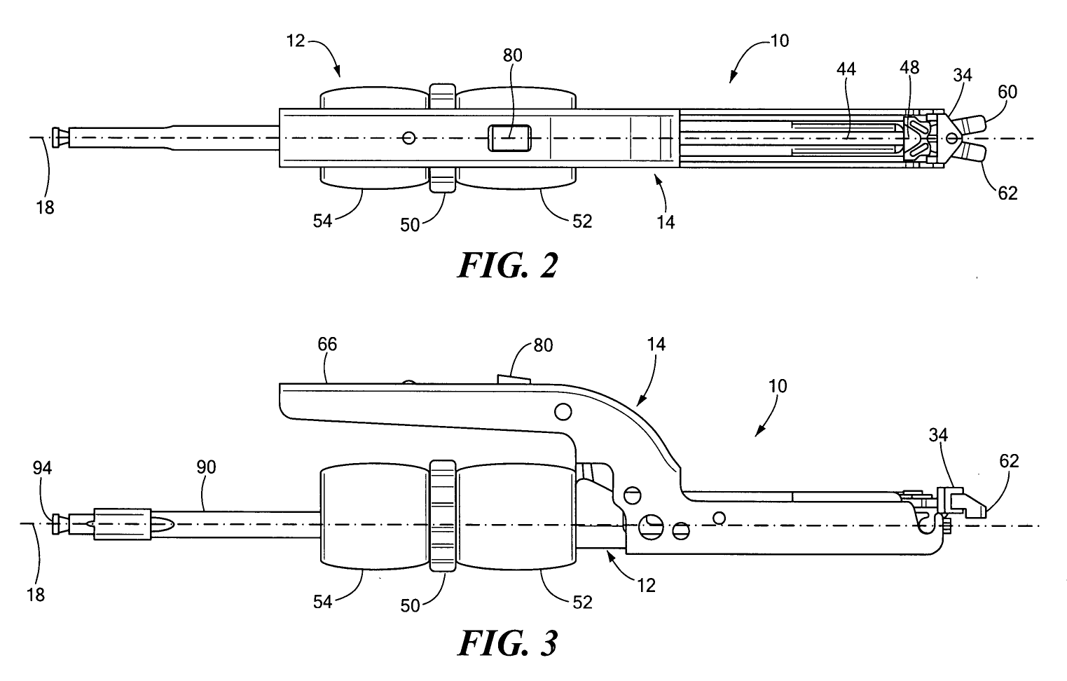

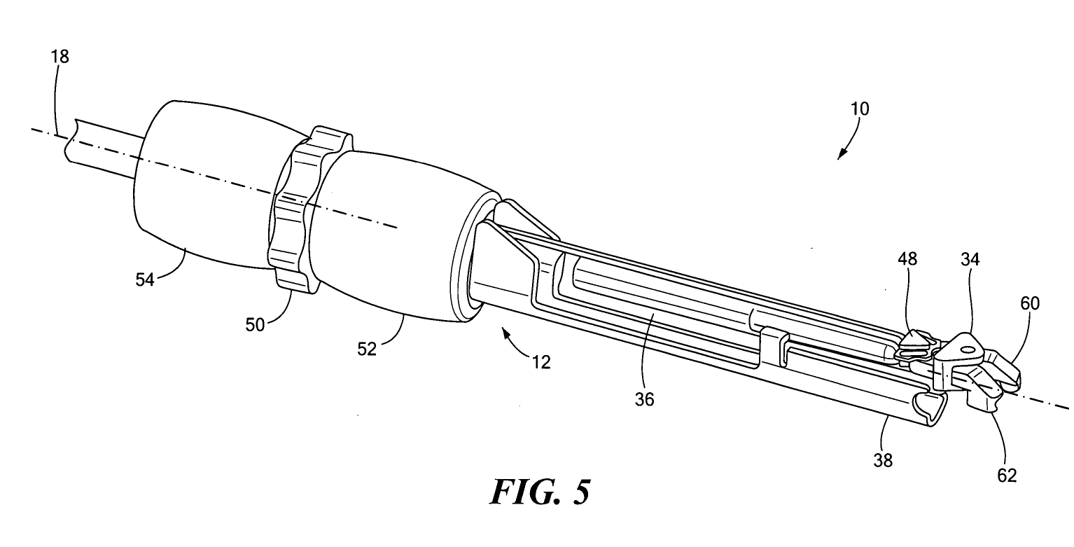

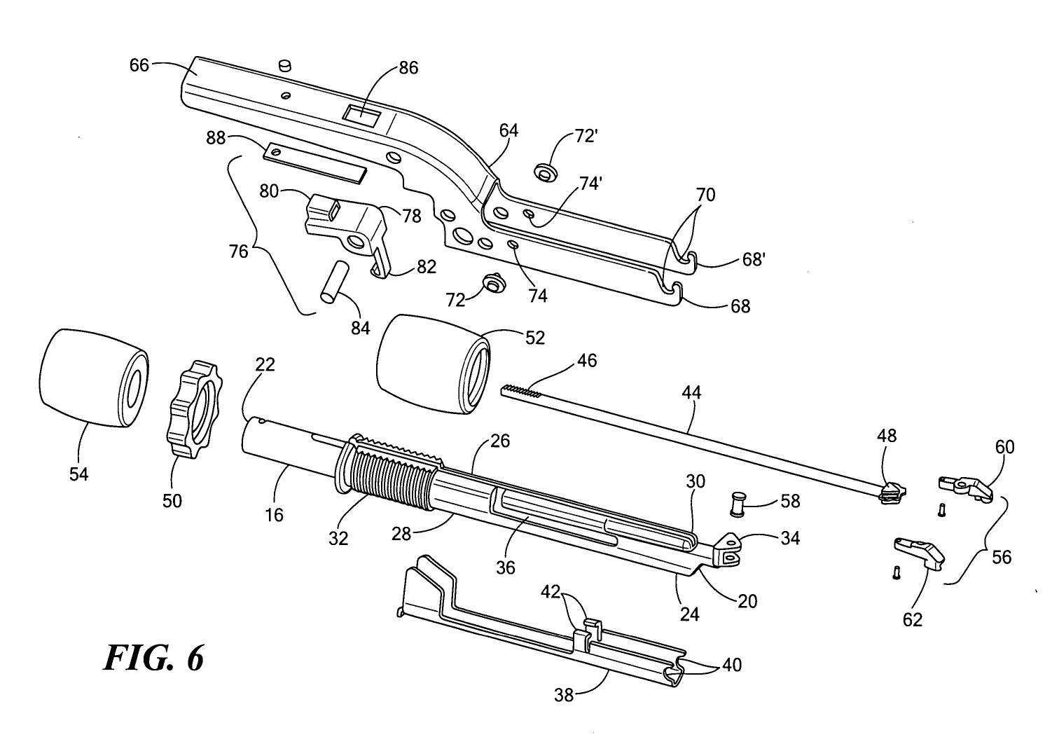

Referring now to the drawing figures in which like reference designators refer to like elements, there is shown in FIGS. 1 through 6, a medical device 10 for rod reduction during orthopedic surgery that is efficient, convenient to use and provides for simultaneous manipulation of a rod and a spinal implant along multiple axes. As shown in FIGS. 1 through 3, the medical device 10 generally includes a shaft assembly 12 and a lever assembly 14 both pivotably coupled about as well as slideably disposed on the shaft assembly 12.

As shown in FIGS. 1 through 6, the shaft assembly 12 can include a shaft 16 defining an elongate body having a longitudinal axis 18, where the shaft 16 may define a hollow channel 20 extending through the entire length of the shaft 16. The shaft 16 may further include both a proximal end 22 and a distal end 24, as well as a top portion 26 and a bottom portion 28. The shaft 16 can also define a longitudinal groove 30 on an exterior surface which extends a substantial length of the top portion 26 of the shaft 16, as well as a threaded portion 32 on the exterior surface near the proximal end 22 of the shaft 16. In addition, the shaft 16 can include a gripper mount 34 located near the distal end 24 of the top portion 26 of the shaft 16. Lever retention grooves 36 may be located on both sides of the shaft assembly 12, where the lever retention grooves 36 define substantially “L” shaped mirrored depressions on the sides of shaft assembly 12.

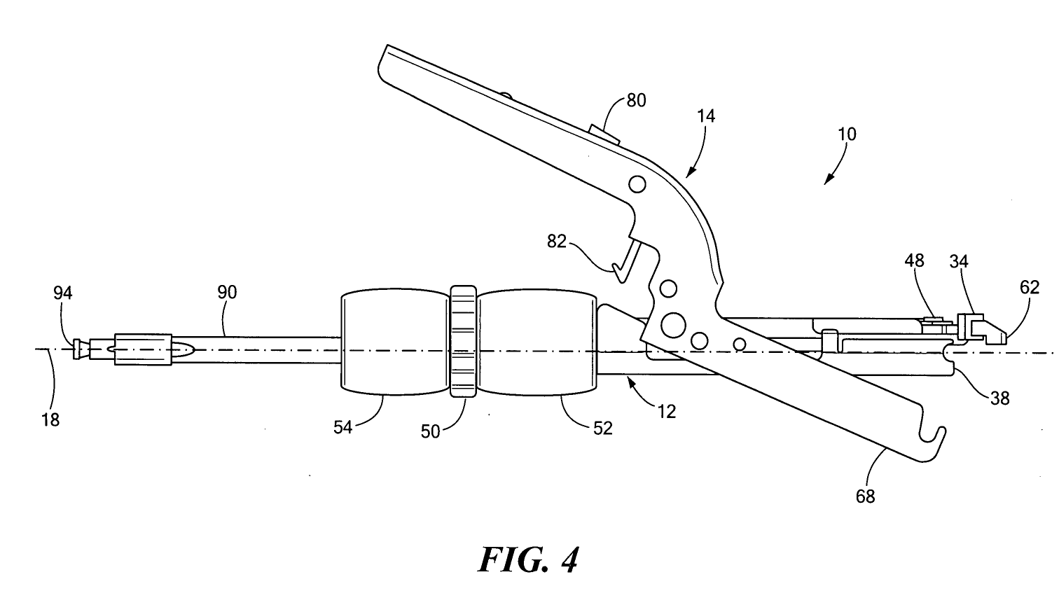

Now referring to FIG. 4 through 6, the shaft assembly 12 also includes a pusher element 38 slideably coupled to the bottom portion 28 of the shaft 16, near the distal end 24. The pusher element 38 defines a generally hollowed, U-shaped body having a substantially semi-circular aperture 40 at an end of the pusher element 38 that is engageable with a spinal rod (not shown). The pusher element 38 may be coupled to the bottom portion 28 of the shaft 16 by a pair of prongs 42 extending from the pusher element 38 which are able to engage a portion of the shaft 16, thereby allowing the pusher element 38 to slide along a length of the shaft 16 in a direction substantially parallel to the longitudinal axis 18 of the shaft 16. Further, the pusher element 38 may be coupled to the shaft 16 such that the semi-circular aperture 40 is positionable at or near the distal end 24 of the shaft 16.

A slide element 44 is included in the shaft assembly 12 of the medical device 10. The slide element 44 defines a generally cylindrical elongate body that is slideably coupled to the top portion 26 of the shaft 16, where the slide element 44 includes a proximal end and a distal end. A threaded portion 46 in the exterior surface of the slide element 44 is located at the proximal end, while the slide element 44 defines a gripper receiving element 48 at the distal end 24. The slide element 44 is positionable within the longitudinal groove 30 of the shaft 16, and may be moved in a direction substantially parallel to the longitudinal axis 18 of the shaft 16.

The shaft assembly 12 also provides a jaw nut 50 rotatably coupled to the proximal end 22 of the shaft 16. The jaw nut 50 defines a substantially hollow cylinder having a threaded inner surface which is engageable with the threaded portion 46 of the slide element 44. By rotating the jaw nut 50, the slide element 44 can be slideably positioned along the length of the shaft 16, while the jaw nut 50 remains in a fixed position with respect to the shaft 16. The jaw nut 50 may further include curved depressions in an outer surface to ease handling and the ability to turn the jaw nut 50 in order to move or otherwise actuate the slide element 44.

The shaft assembly 12 of the present invention further provides a first shaft grip 52 rotatably coupled to the threaded portion 32 of the shaft 16. The first shaft grip 52 defines a generally hollow cylindrical body, where an interior surface of the first shaft grip 52 is threaded in order to rotatably advance or retract along the threaded portion 32 of the shaft 16, thereby allowing the first shaft grip 52 to be movably positioned towards either the distal end 24 or proximal end 22 of the shaft 16. Furthermore, the first shaft grip 52 is coupled to the shaft 16 as to abut or otherwise engage the pusher element 38, such that by rotating the first shaft grip 52 about the threaded portion 32 of the shaft 16, both the first shaft grip 52 and the pusher element 38 can be slideably positioned along the length of the shaft 16 in a direction substantially parallel to the longitudinal axis 18 of the shaft 16. A second shaft grip 54 may also be coupled to the shaft 16 to ease the handling and grasping of the medical device 10, to secure the jaw nut 50 in place, or the like.

A gripper assembly 56 is provided which is pivotably coupled to the gripper mount 34 at the distal portion of the shaft 16. The gripper assembly 56 can be pivotably affixed to the gripper mount 34 and thus the shaft 16 through the use of a pivot rod 58. The gripper assembly 56 may include a first gripper element 60 and a second gripper element 62, where the first and second gripper elements 60,62 are engageable with an implant or pedicle screw (not shown). In addition, the first and second gripper elements 60,62 may be movably coupled with the gripper receiving element 48 of the slide element 44, whereby manipulation of the slide element 44 can cause the first and second gripper elements 60,62 to either extend, retract, pivot outwards, or pivot inwards, further facilitating the secure engagement of a surgical implant.

Still referring to FIGS. 4 through 6, the lever assembly 14 is both pivotably coupled to as well as slideably disposed on the shaft assembly 12. The lever assembly 14 can include a lever element 64 and a lever grip 66. The lever element 64 can include two lever arms 68,68′ having a distal portion defining a rod channel 70 configured to receive a rod (not shown) therein. The lever element 64 can further include a set of pivots 72,72′ for insertion through lever element pivot holes 74,74′ in order to couple the lever assembly 14 to the lever retention grooves 36 located on either side of the shaft assembly 12. By coupling to the lever retention grooves 36, the lever assembly 14 can both pivot about the shaft assembly 12, as well as be slideably positioned along a length of the shaft assembly 12 in a direction substantially parallel to the longitudinal axis 18 of the shaft 16. In operation, the lever assembly 14 is removable such that if reduction of the rod is only required in a single plane, the lever assembly 14 can be easily detached from the medical device 10, thereby providing enhanced tool and use simplification.

The lever assembly 14 may further include a latch mechanism 76, where the latch mechanism 76 includes a latch 78 pivotably affixed to the handle portion of the lever assembly 14. The latch 78 includes a trigger element 80, which can be in the form of a button or raised surface, as well as a hook 82 able to engage a portion of the shaft assembly 12. The latch 78 may be pivotably affixed to the lever assembly 14 through the use of a pin 84 extending through both the latch 78 and the lever assembly 14, such that the trigger element extends through an aperture 86 in the lever grip 66, while the hook 82 is able to engage a portion of the first shaft grip 52, for example. In addition, the latch mechanism 76 may include a biasing element 88 that biases the latch 78 towards a particular position when not engaged, i.e., the hook 82 remains in position to readily engage the shaft assembly 12 when the lever assembly 14 is moved. The latch mechanism 76 allows the lever assembly 14 to be releasably secured to the shaft assembly 12 without the need for additional force or pressure to be applied to the lever assembly 14. Upon subsequent need to reposition or otherwise manipulate the lever assembly 14, the latch mechanism 76 may be disengaged from the shaft assembly 12, allowing the lever assembly 14 to thereby pivot and slide about the shaft assembly 12.

The medical device 10 of the present invention may further provide a driver assembly 90. The driver assembly may include an elongated shaft 92 having an implant-engaging end (not shown), as well as a tool-engaging end 94. The implant-engaging end of the shaft 92 can be used to tighten a set screw or other fastening mechanism of a surgical implant, while the tool-engaging end 94 can be coupled to a power tool or hand tool to ease turning of the shaft 16. The driver assembly 90 is positionable and extendable through the hollow channel 20 of the shaft 16 of the medical device 10.

In an exemplary use of the present device, the medical device 10 of the present invention can aid in the multi-planar positioning of a spinal fixation rod within a bone fastener such as a pedicle screw or the like during a surgical procedure. For example, by manipulating the jaw nut 50 and thus the slide element 44, the gripper assembly 56 can be positioned and manipulated to securely engage an exposed portion of the pedicle screw. Subsequently, the lever assembly 14 may be used to engage the spinal fixation rod by positioning the two lever arms 68,68′ such that the spinal fixation rod is received in the rod channel 70 of the lever arms 68,68′. By applying force to the lever grip 66, the spinal fixation rod can be moved towards the distal end 24 of the shaft assembly 12, where the pedicle screw has previously been engaged. Once the spinal fixation rod is in an appropriate position, the first shaft grip 52 can be turned to slideably move the pusher element 38 such that the aperture 40 in the pusher element 38 engages the spinal fixation rod as well. Through the combined manipulation of the pusher element 38 along the longitudinal axis 18 of the shaft assembly 12, the pivoting force applied transversely to the longitudinal axis 18 by the lever assembly 14, and turning the entire medical device 10 about the longitudinal axis 18, the spinal fixation rod can be maneuvered in all three planes of motion into the desired position within the pedicle screw, and the latch mechanism 76 of the lever assembly 14 can be engaged to hold the spinal fixation rod in place. Once the rod has been inserted into the pedicle screw, it can be securely fastened by a setscrew or other affixation means using the driver assembly 90, if necessary.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device for reducing a rod towards a bone fastener, comprising:
a shaft having a proximal end, a distal end, and a longitudinal axis;
a gripper assembly coupled to the distal end of the shaft, the gripper assembly being engageable with the bone fastener;
a pusher element slideably coupled to the shaft, the pusher element being engageable with the rod; and
a lever element pivotably coupled to the shaft, the lever element being engageable with the rod.

2. The medical device according to claim 1, wherein the lever element is further movable along a length of the shaft in a direction substantially parallel to the longitudinal axis of the shaft.

3. The medical device according to claim 1, wherein the pusher element is slideable in a direction substantially parallel to the longitudinal axis of the shaft.

4. The medical device according to claim 1, wherein the gripper assembly includes a first gripper element and a second gripper element.

5. The medical device according to claim 4, wherein the first and second gripper elements are pivotable with respect to each other.

6. The medical device according to claim l, wherein the pusher element defines an aperture able to receive at least a portion of the rod.

7. The medical device according to claim 1, wherein the lever element defines first and second lever arms.

8. The medical device according to claim 7, wherein the first and second lever arms define a rod channel able to receive at least a portion of the rod.

9. The medical device according to claim 1, further comprising a latch mechanism able to releasably secure the lever element to the shaft assembly.

10. The medical device according to claim 1, further comprising a slide element defining a threaded portion, the slide element being slideably coupled to the shaft and further coupled to the gripper assembly.

11. The medical device according to claim 10, further comprising a jaw nut coupled to the shaft, the jaw nut being rotatably engaged with the threaded portion of the slide element.

12. The medical device according to claim 1, wherein the shaft further defines a threaded portion.

13. The medical device according to claim 12, further comprising a shaft grip rotatably engaged with the threaded portion of the shaft, the shaft grip abutting at least a portion of the pusher element.

14. A medical device for reducing a rod towards a bone fastener, comprising:
a shaft assembly having a proximal end and a distal end;
a gripper assembly coupled to the distal end of the shaft, wherein the gripper assembly includes first and second gripper elements engageable with the bone fastener;
a pusher element slideably coupled to the shaft, wherein the pusher element defines an aperture able to receive at least a portion of the rod; and
a lever assembly pivotably coupled to the shaft, wherein the lever assembly includes first and second lever arms defining a rod channel able to receive at least a portion of the rod.

15. The medical device according to claim 14, further comprising a slide element slideably coupled to the shaft, the slide element further being coupled to the gripper assembly;

16. The medical device according to claim 14, wherein the lever assembly is slideable along a length of the shaft.

17. The medical device according to claim 14, wherein the lever assembly further includes a latch mechanism for releasably securing the lever assembly to the shaft.

18. A medical device for reducing a rod towards a bone fastener, comprising:
a shaft defining a proximal end, a distal end, a top portion, and a bottom portion, the shaft further defining a first threaded portion;
a gripper assembly coupled to the distal end of the shaft, wherein the gripper assembly is engageable with the bone fastener;
a slide element slideably disposed on the top portion of the shaft and coupled to the gripper assembly, the slide element defining a second threaded portion;
a pusher element slideably coupled to the bottom portion of the shaft, wherein the pusher element defines an aperture engageable with the rod;
a shaft grip rotatably engaged with the first threaded portion of the shaft, the shaft grip abutting at least a portion of the pusher element;
a jaw nut coupled to the shaft, the jaw nut rotatably engaged with the second threaded portion of the slide element;
a lever assembly pivotably coupled to the shaft, the lever assembly further being slideably positionable on the shaft, wherein the lever assembly defines a lever grip and first and second lever arms, wherein the first and second lever arms define a rod channel engageable with the rod, and wherein the lever assembly further includes a latch mechanism for releasably securing the lever assembly to the shaft.

19. The medical device according to claim 14, wherein the gripper assembly includes a first and second gripper element.
